(19) United States
(12) Patent Application Publication
(10) Pub. No.: US 2025/0009605 A1
(43) Pub. Date: Jan. 9, 2025

(54) NIPPLE SHIELD WITH PORT AND FLAP-COVERED CHANNEL TO SUPPLEMENT LACTATION DURING BREASTFEEDING

(71) Applicant: Neotech Products LLC, Valencia, CA (US)

(72) Inventors: Yekaterina Spivak, Mountainside, NJ (US); Maksim Spivak, Mountainside, NJ (US); Aaron Bronshtein, Oakland, NJ (US); Michael Maloney, Caledon East (CA)

(73) Assignee: Neotech Products LLC, Valencia, CA (US)

(21) Appl. No.: 18/826,922

(22) Filed: Sep. 6, 2024

Related U.S. Application Data
(62) Division of application No. 16/830,098, filed on Mar. 25, 2020, now Pat. No. 12,083,074.

(51) Int. Cl. A61J 13/00; A61J 11/00; A61J 15/00; A61M 1/06

(52) U.S. Cl. CPC A61J 13/00; A61J 11/0005; A61J 15/0053; A61M 1/068

(57) ABSTRACT

Nipple shields can be used by breastfeeding mothers to facilitate the feeding process. The disclosed embodiments relate to nipple shields with a port through which liquid supplement can flow from a supply tube into an interior section of the shield. The liquid supplement can be drawn through ports in a tip of the shield in order to augment a naturally expressed flow of milk. A flap of material is bonded to an interior portion of the nipple shield over an optional channel formed in the interior portion so as to form a closed, flap-covered channel leading from an input port on a lower portion of the shield to a an interior location near the tip of the shield.

### RELATED APPLICATIONS

The subject matter of this application is related to U.S. Provisional Application No. 62/823,412, filed on 2019 Mar. 25, and to U.S. Design patent application Ser. No. 29/683,891, filed on 2019 Mar. 15, all of which applications are incorporated herein by reference in their entireties.

### BACKGROUND OF THE INVENTION

Nipple shields can be used by breastfeeding mothers to facilitate the feeding process.

### SUMMARY OF THE INVENTION

The disclosed embodiments relate to nipple shields with a port through which liquid supplement can flow from a supply tube into an interior section of the shield. The liquid supplement can be drawn through ports in a tip of the shield in order to augment a naturally expressed flow of milk. A flap of material is bonded to an interior portion of the nipple shield over an optional channel formed in the interior portion so as to form a closed, flap-covered channel leading from an input port on a lower portion of the shield to a an interior location near the tip of the shield.

### BRIEF DESCRIPTION OF THE DRAWINGS

FIGS. 1-14 illustrate an assembled nipple shield with a flap that is bonded in place according to a first embodiment. FIGS. 15-28 illustrate a partially assembled nipple shield with a loose flap that has not yet been bonded in place according to a second embodiment. The first and second embodiments are at least partially transparent as indicated in the figures. FIGS. 29-34 illustrate an assembled nipple shield that is generally opaque as indicated in the figures according to a third embodiment.

In the drawings, broken lines illustrate primarily environmental structure that is nevertheless within the scope of the present disclosure. The dot-dash broken lines in the drawings delineate different portions of the embodiments and can be ignored since they do not represent any particular structure or markings thereon. The short-short-long dash broken lines identify cross section views and enlarged views of the embodiments.

FIG. 1 is a perspective view.

FIG. 2 is a top view (exterior).

FIG. 3 is a bottom view (interior).

FIG. 4 is front view (opposite the port and tube).

FIG. 5 is a back view (proximate the port and tube).

FIG. 6 is a right side view, and the left side view (not illustrated) is a mirror image of the right side view.

FIG. 7 is a cross-section view along the short-short-long dash line indicated in FIG. 2.

FIG. 8 is an enlarged view of the encircled area indicated in FIG. 7.

FIG. 9 is a cross-section view along the correspondingly numbered short-short- long dash line indicated in FIG. 7.

FIG. 10 is a cross-section view along the correspondingly numbered short- short-long dash line indicated in FIG. 7.

FIG. 11 is a cross-section view along the correspondingly numbered short- short-long dash line indicated in FIG. 7.

FIG. 12 is an enlarged view of the encircled area indicated in FIG. 9.

FIG. 13 is an enlarged view of the encircled area indicated in FIG. 10.

FIG. 14 is an enlarged view of the encircled area indicated in FIG. 11.

FIG. 15 is a perspective view.

FIG. 16 is a top view (exterior).

FIG. 17 is a bottom view (interior).

FIG. 18 is front view (opposite the port and tube).

FIG. 19 is a back view (proximate the port and tube).

FIG. 20 is a right side view, and the left side view (not illustrated) is a mirror image of the right side view.

FIG. 21 is a cross-section view along the short-short-long dash line indicated in FIG. 16.

FIG. 22 is an enlarged view of the encircled area indicated in FIG. 21.

FIG. 23 is a cross-section view along the correspondingly numbered short- short-long dash line indicated in FIG. 21.

FIG. 24 is a cross-section view along the correspondingly numbered short- short-long dash line indicated in FIG. 21.

FIG. 25 is a cross-section view along the correspondingly numbered short- short-long dash line indicated in FIG. 21.

FIG. 26 is an enlarged view of the encircled area indicated in FIG. 23.

FIG. 27 is an enlarged view of the encircled area indicated in FIG. 24.

FIG. 28 is an enlarged view of the encircled area indicated in FIG. 25.

FIG. 29 is a perspective view.

FIG. 30 is a top view (exterior).

FIG. 31 is a bottom view (interior).

FIG. 32 is front view (opposite the port and tube).

FIG. 33 is a back view (proximate the port and tube).

FIG. 34 is a right side view, and the left side view (not illustrated) is a mirror image of the right side view.

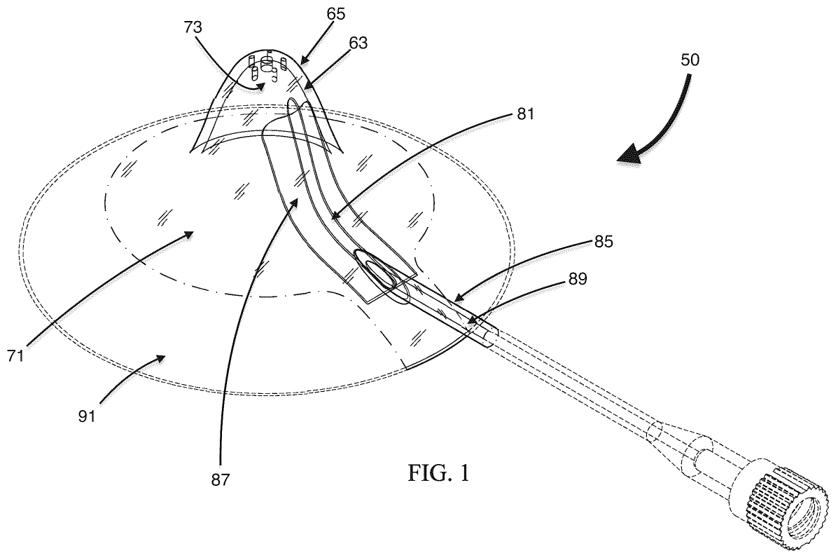

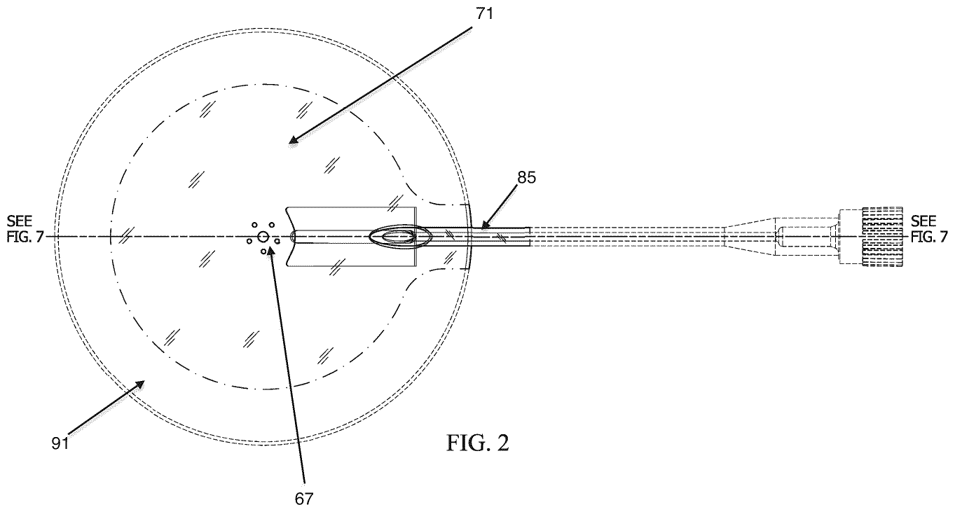

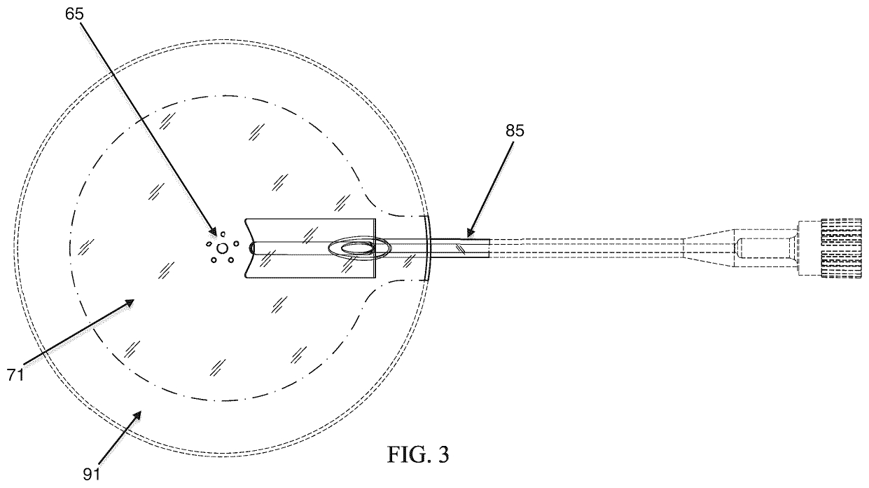

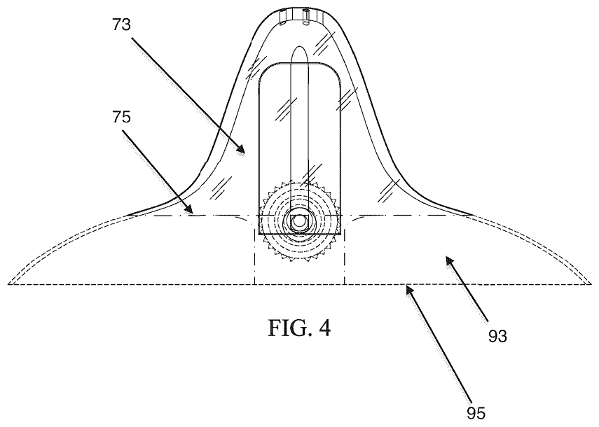

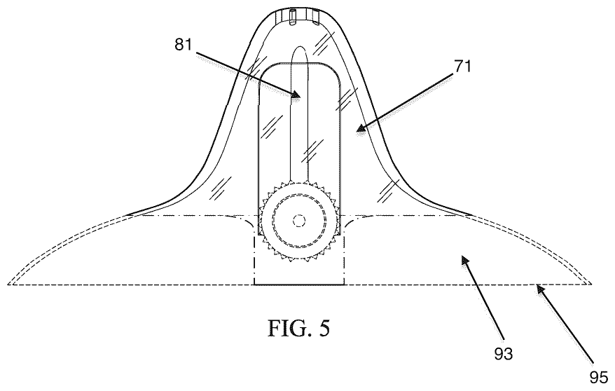

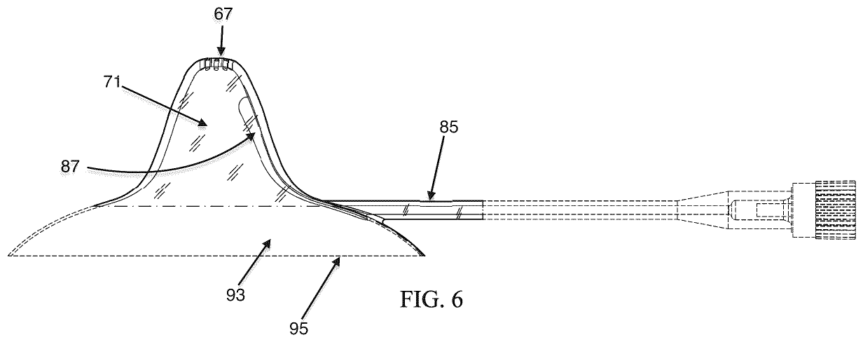

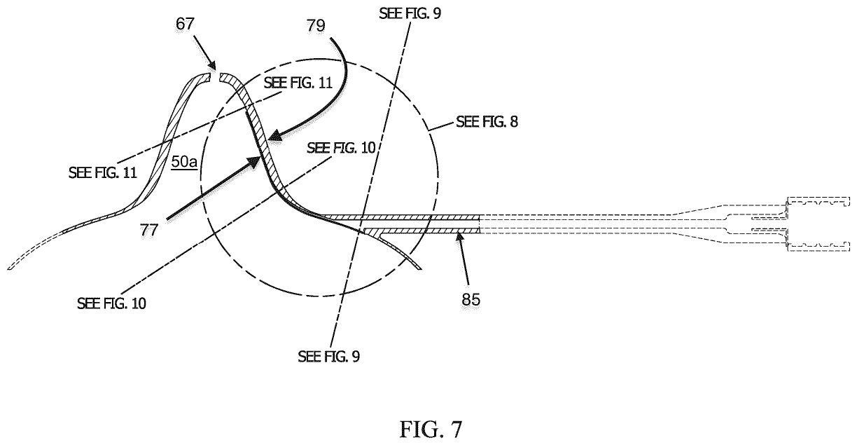

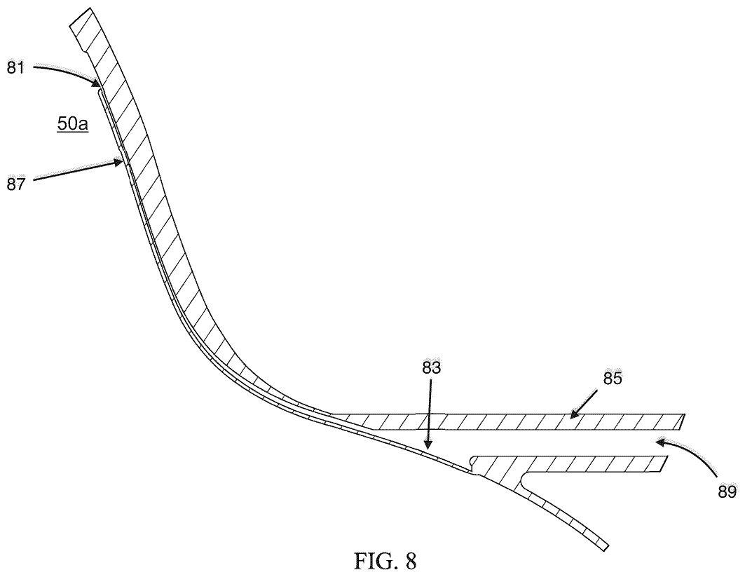

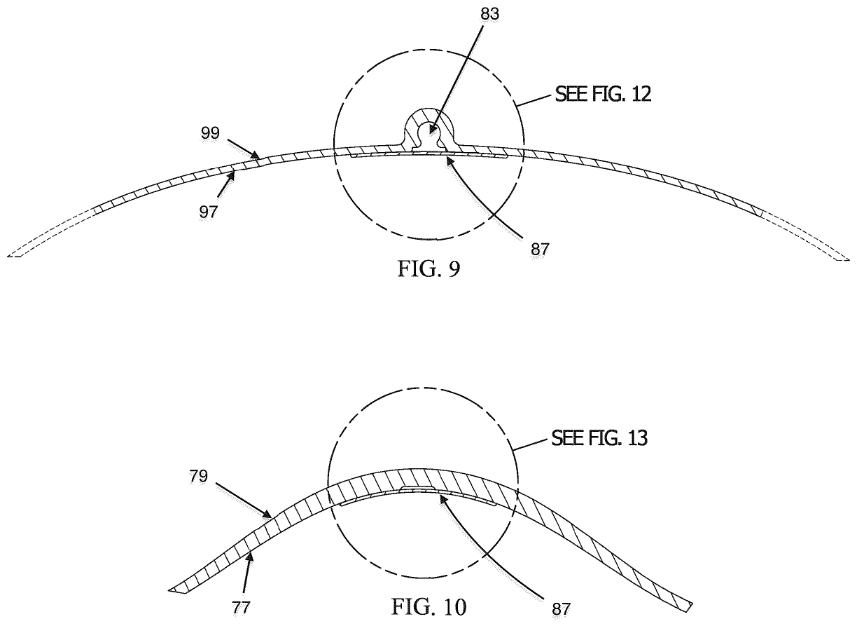

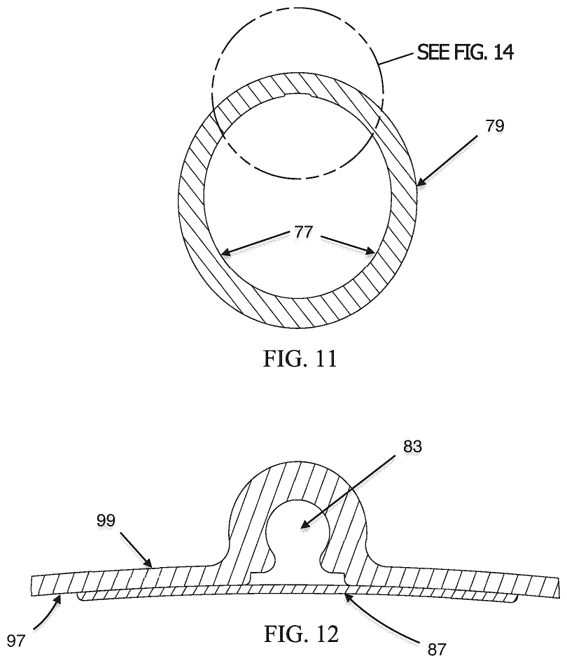

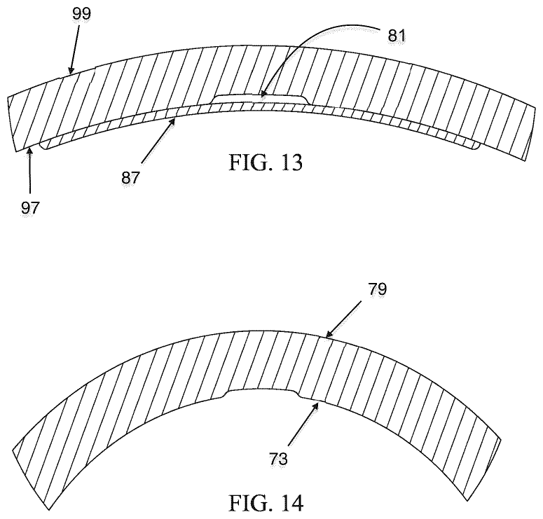

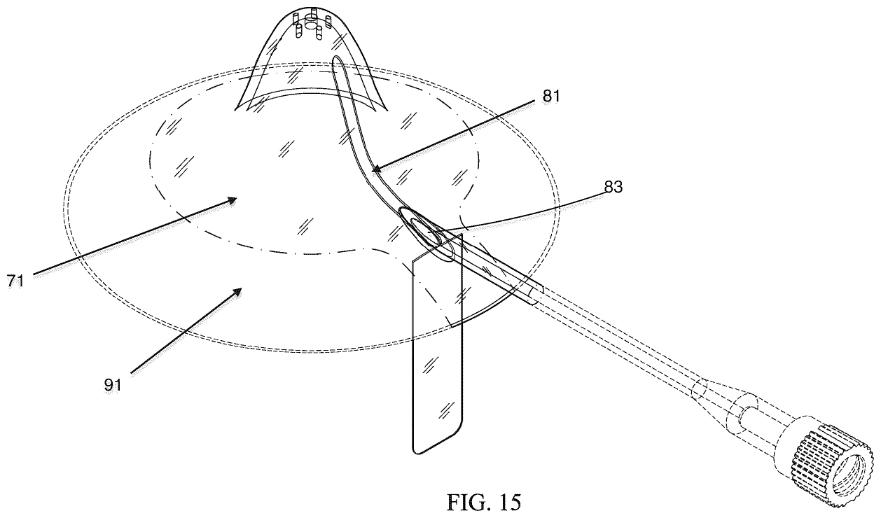

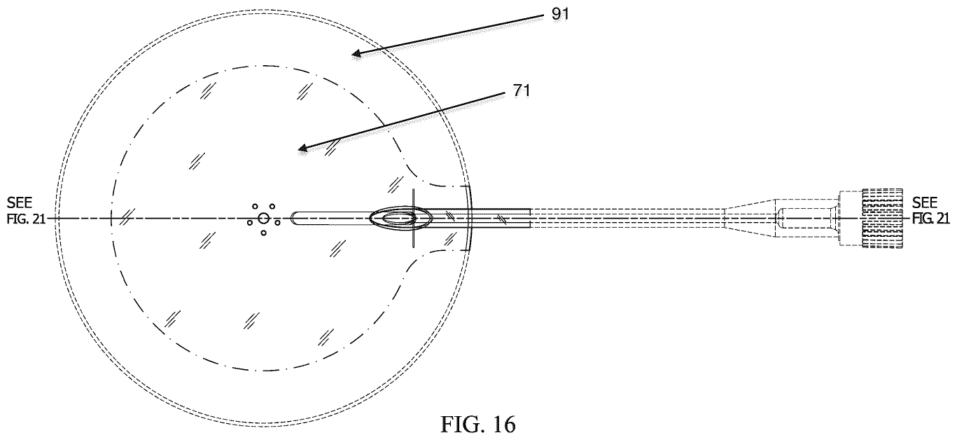

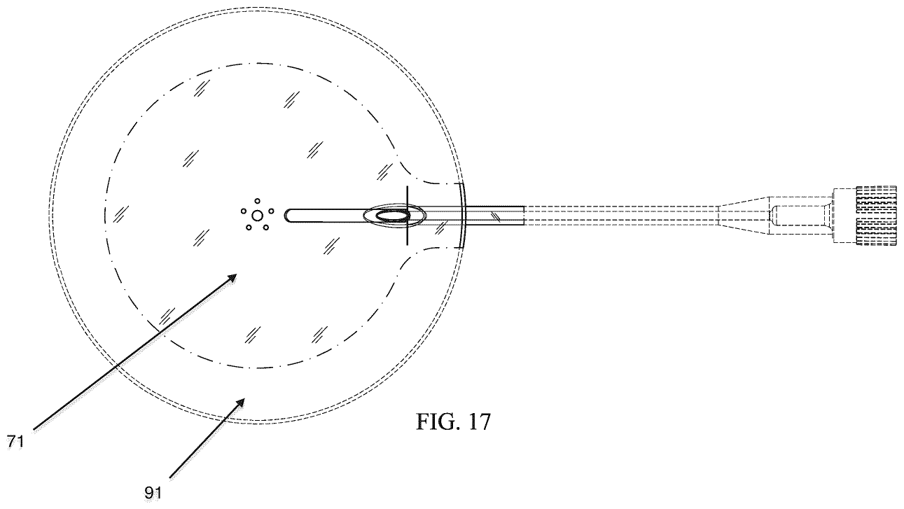

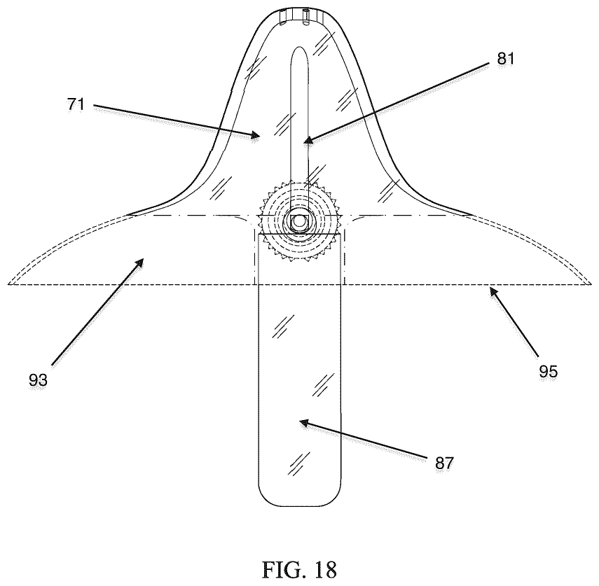

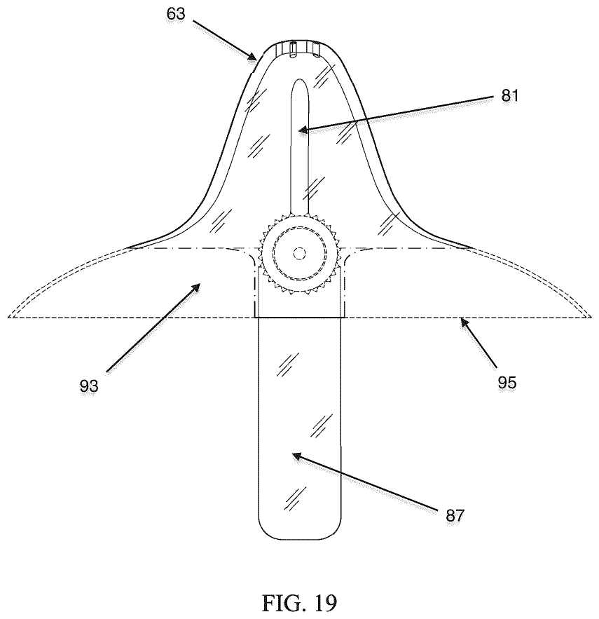

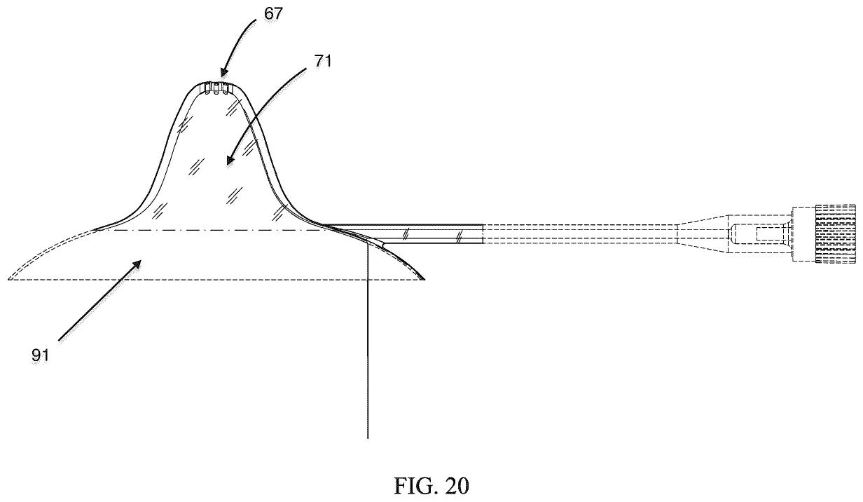

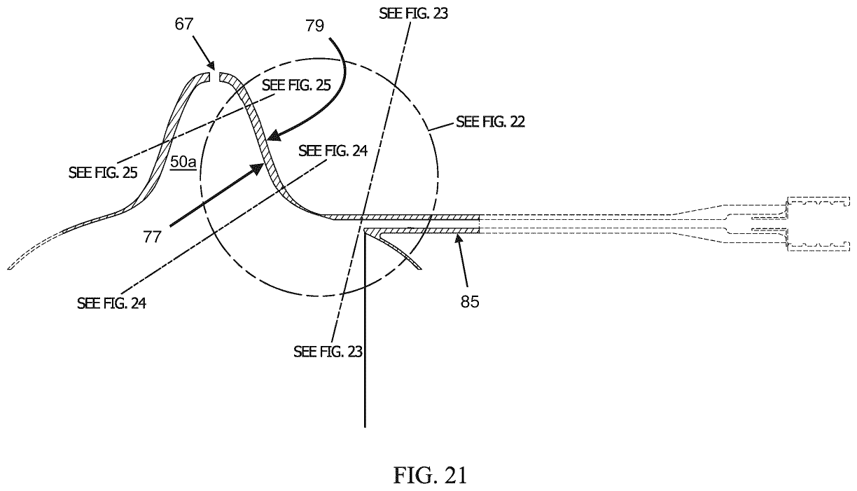

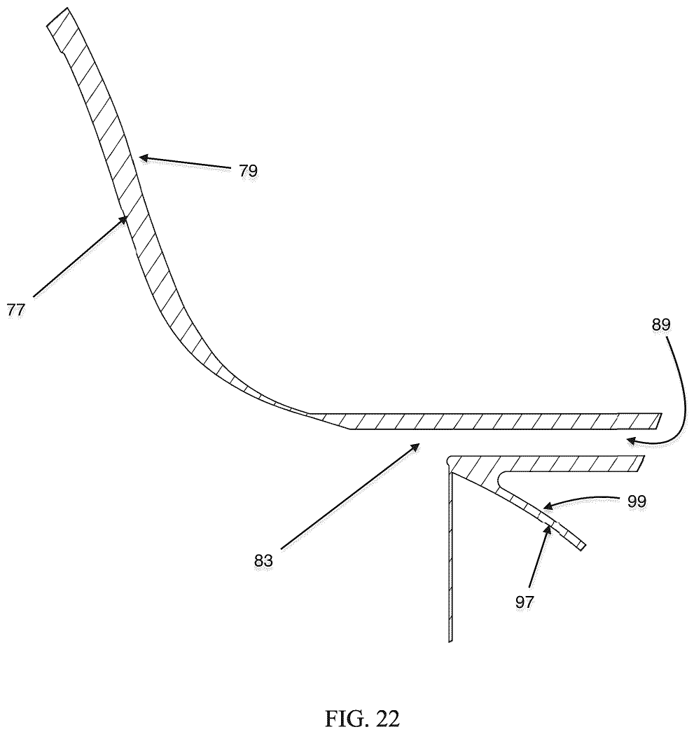

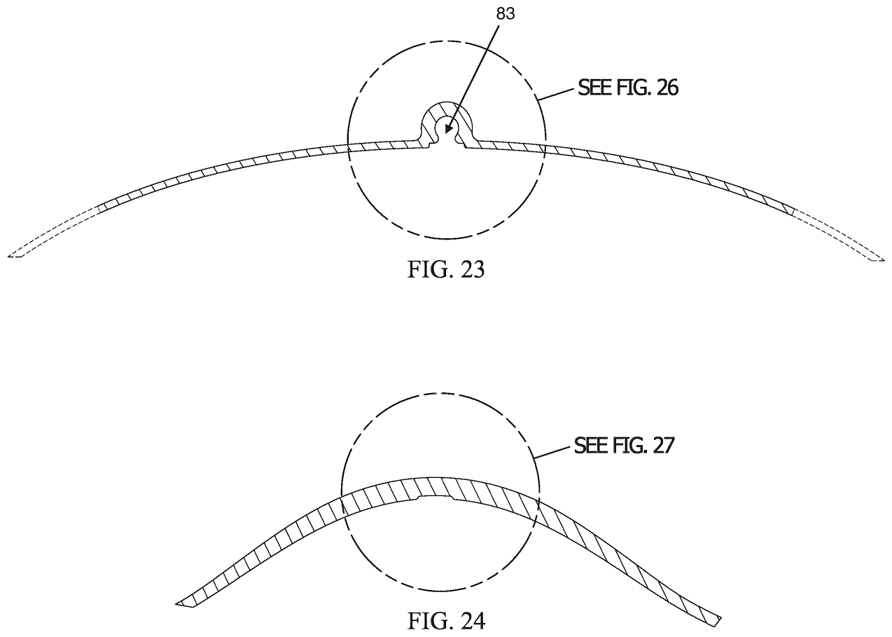

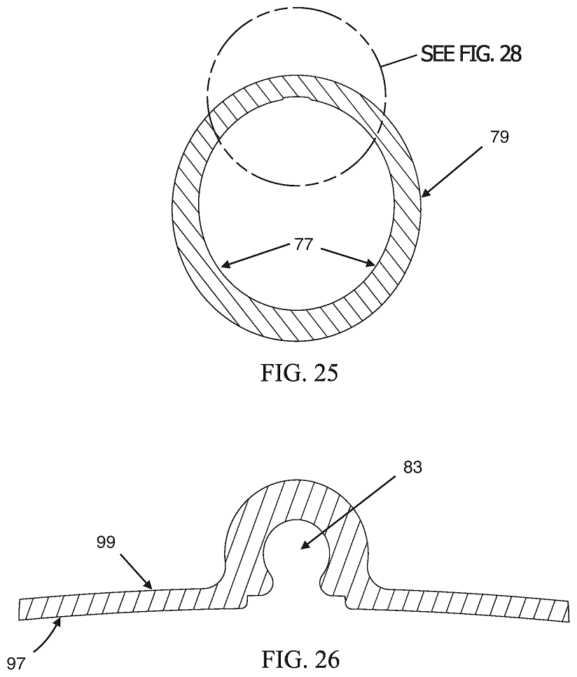

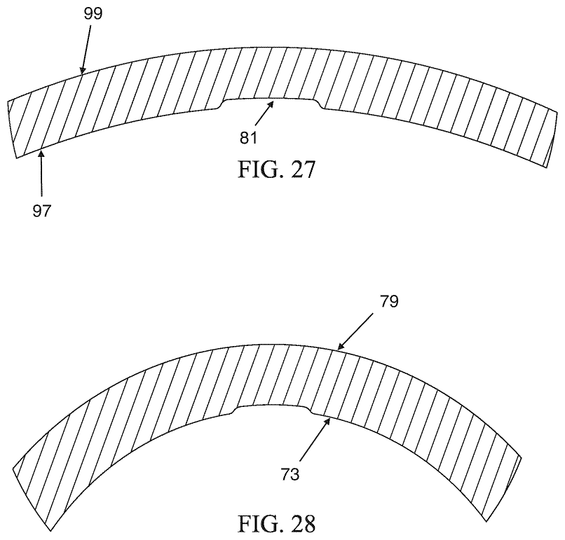

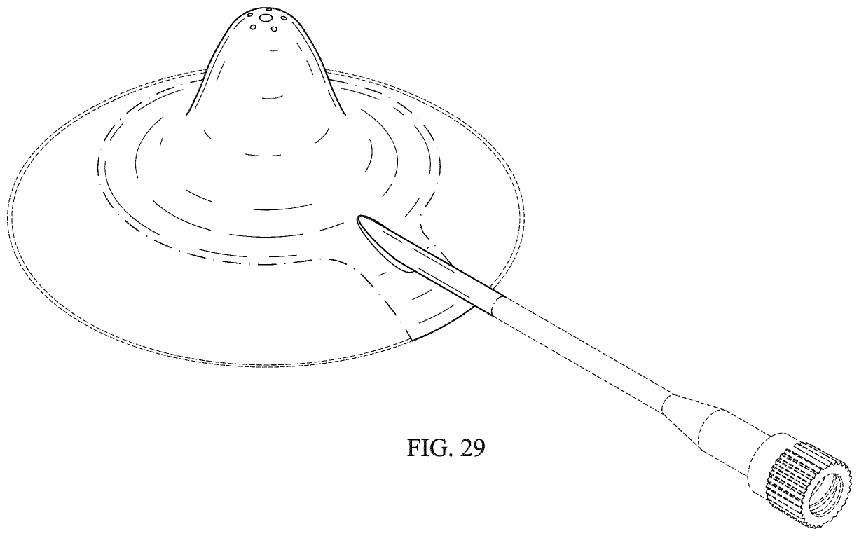

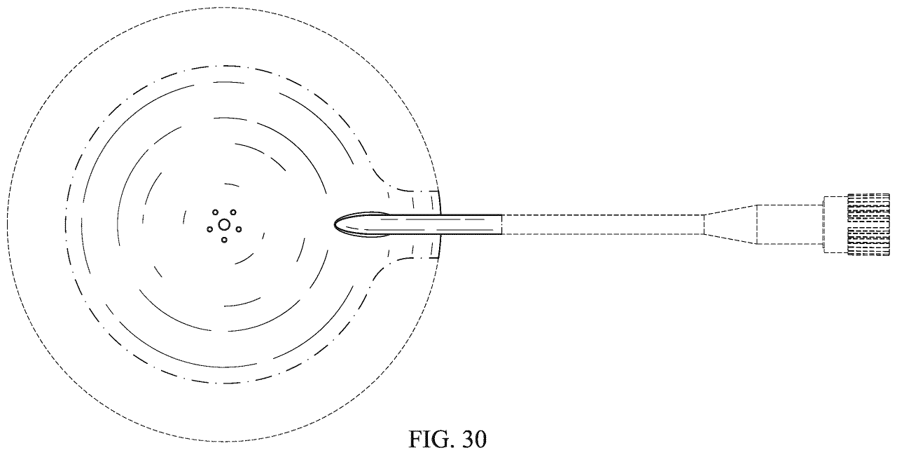

### DETAILED DESCRIPTION

In the following description, references are made to various embodiments in accordance with which the disclosed subject matter can be practiced. Some embodiments may be described using the expressions one/an/another embodiment or the like, multiple instances of which do not necessarily refer to the same embodiment. Particular features, structures or characteristics associated with such instances can be combined in any suitable manner in various embodiments unless otherwise noted. By way of example, this disclosure may set out a set or list of a number of options or possibilities for an embodiment, and in such case, this disclosure specifically contemplates all clearly feasible combinations and/or permutations of items in the set or list.

Nipple shields can be used by breastfeeding mothers to facilitate the feeding process. In accordance with various embodiments, a nipple shield can include a port through which liquid supplement can flow from a supply tube into an interior section of the shield. The liquid supplement can be drawn through ports in a tip of the shield in order to augment a naturally expressed flow of milk.

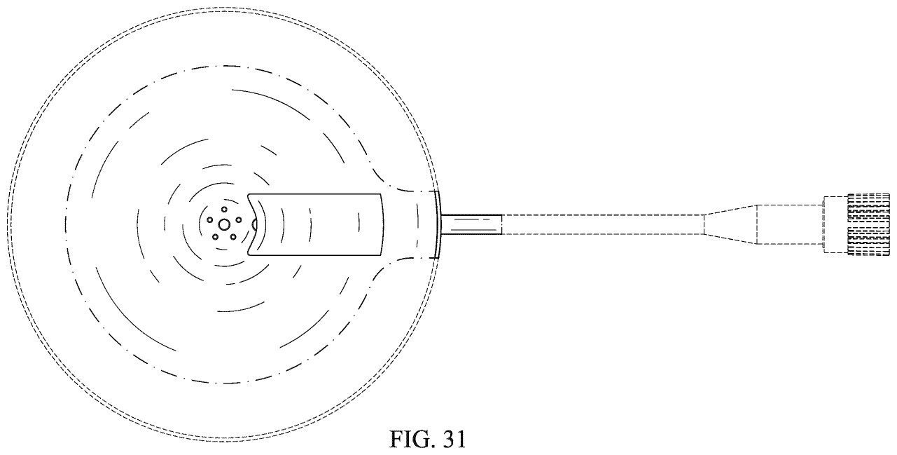

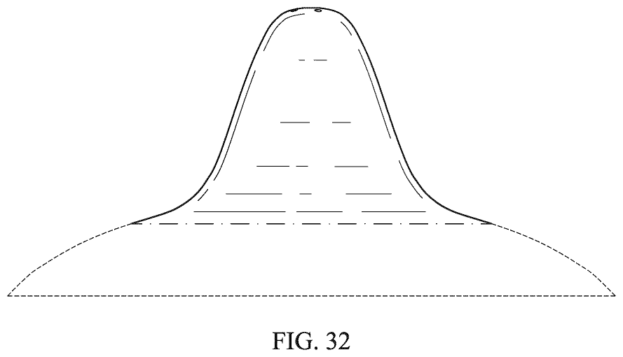

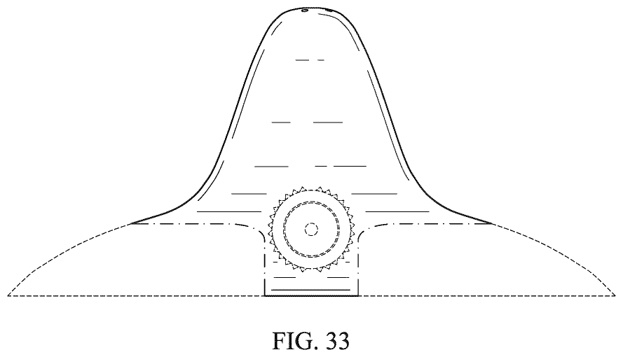

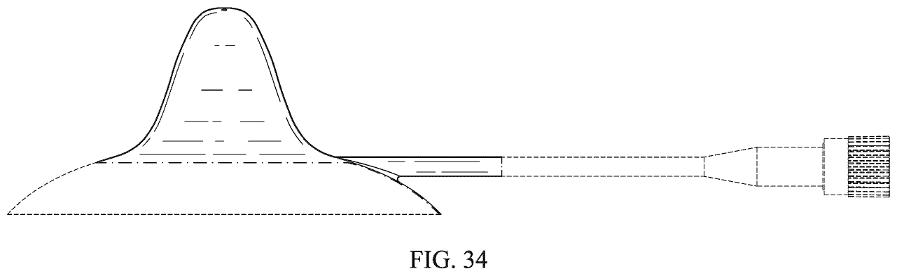

A flap of material is shown extending from the shield in the second embodiment, and that is bonded, in the assembled first embodiment, to an interior portion of the nipple shield over a channel formed in the interior portion of the shield so as to form a closed channel leading from the port to a location near an interior tip of the shield. The flap of material is also shown bonded in place in the third embodiment in FIG. 31.

In accordance with various embodiments, a nipple shield can include the following features. The nipple shield can include a tip section having an interior surface, an exterior surface and one or more perforations passing through the tip section between the interior surface and the exterior surface. The nipple shield can include a substantially frustoconical-shaped middle section having an upper portion adjoining the tip section and a lower portion opposite the upper portion, the upper portion having a diameter less than the lower portion, the middle section having a substantially frustoconical-shaped interior surface that adjoins the interior surface of the tip section, the middle section having a substantially frustoconical-shaped exterior surface that adjoins the exterior surface of the tip section, the middle section having an open channel formed along the interior surface and extending from the lower portion to the upper portion of the middle section. The nipple shield can include a lower section having an upper portion adjoining the middle section and a lower portion opposite the upper portion, the lower section having an interior surface that adjoins the interior surface of the middle section, the lower section having an exterior surface that adjoins the exterior surface of the middle section, the lower section having an open port passing between the interior surface and the exterior surface. The nipple shield can include a supply tube adjoining the lower section at the open port forming a passage through the supply tube to the interior surface of the lower section. The nipple shield can include a channel cover of thin flexible sheet material, the channel cover being bonded, joined, affixed or sealed to portions of the interior surface of the lower section to cover the open port, the channel cover being bonded, joined, affixed or sealed to portions of the interior surface of the middle section to cover a portion of the open channel, wherein the bonded, joined, affixed or sealed portions of the channel cover extend along an interior surface of the nipple shield from the open port and along two sides of the channel to a location proximate the tip section.

In one embodiment, the interior surface of the tip section is concave and the exterior surface of the tip section is convex.

In one embodiment, the tip section, the middle section and the lower section are formed of silicone rubber.

In one embodiment, the tip section, the middle section, the lower section, the supply tube, and the channel cover are formed of silicone rubber.

In one embodiment, the channel cover is formed of a flap of material that is molded contiguously with at least the lower section, and wherein the molded flap is subsequently bonded to the middle section along two sides of the channel.

In one embodiment, the channel is omitted from the shield, such that the interior surface of the middle section does not include a channel, such that the channel cover is not bonded to the interior of the middle section along a path where the channel would otherwise be located, and such that a stream of supplement can create a gap and flow under pressure in an unbonded area between the channel cover and the interior surface of the middle section.

Although the subject matter has been described in terms of certain embodiments, other embodiments that may or may not provide various features and aspects set forth herein shall be understood to be contemplated by this disclosure. The specific embodiments described above are disclosed as examples only, and the scope of the patented subject matter is defined by the claims that follow. In the claims, a portion shall include greater than none and up to the whole of a thing.

1-6. (canceled).

7. A nipple shield comprising:
a tip section having an interior tip surface, an exterior tip surface and at least one perforation passing through the tip section between the interior tip surface and the exterior tip surface;
a substantially frustoconical-shaped middle section having a middle section upper portion adjoining the tip section and a middle section lower portion opposite the middle section upper portion, the middle section upper portion having a diameter less than the middle section lower portion, the middle section having a substantially frustoconical-shaped interior middle surface that adjoins the interior tip surface, the middle section having a substantially frustoconical-shaped exterior middle surface that adjoins the exterior tip surface;
a lower section having a lower section upper portion adjoining the middle section and a lower section lower portion opposite the lower section upper portion, the lower section having an interior lower surface that adjoins the interior middle surface, the lower section having an exterior lower surface that adjoins the exterior middle surface, the lower section having an open port passing between the interior lower surface and the exterior lower surface;
a supply tube adjoining the lower section at the open port forming a passage through the supply tube to the interior lower surface; and
a cover of flexible sheet material disposed on the interior lower surface and the interior middle surface so as to cover the open port, which cover extends from proximate to the open port in the lower section to proximate to the at least one perforation of the tip section, the cover being attached to portions of the interior lower surface proximate to the open port.

8. The nipple shield of claim 7, wherein the cover is configured such that a stream of supplement from the supply tube can create a gap and flow under pressure from the open port to the tip section in an unattached area between the cover and the interior surface of the middle section.

9. The nipple shield of claim 7, wherein the interior tip surface is concave and the exterior tip surface is convex.

10. The nipple shield of claim 7, wherein the tip section, the middle section and the lower section are formed of silicone rubber.

11. The nipple shield of claim 7, wherein the tip section, the middle section, the lower section, the supply tube, and the cover are formed of silicone rubber.

12. The nipple shield of claim 7, wherein the cover is formed of a flap of material that is molded contiguously with at least the lower section, and wherein the molded flap is subsequently attached to the middle section along two side edges of the cover.

13. A nipple shield comprising:
a substantially frustoconical-shaped body having a lower section, a middle section, and a tip section, wherein the middle section has a diameter less than the lower section and the tip section has a diameter less than the middle section;
said frustoconical-shaped body being made of a rubber material and having an interior surface and an exterior surface spanning each of the lower section, the middle section, and the tip section;
at least one perforation in the tip section passing from the interior surface to the exterior surface;
an open port in the lower section passing from the exterior surface to the interior surface and a supply tube adjoining the exterior surface of the lower section so as to be in fluid communication with the open port; and
a cover of flexible sheet material on the interior surface disposed over the open port and extending from proximate to the open port of the lower section to proximate to the tip section, wherein the cover is attached to the interior surface of the lower section and the interior surface of the middle section along both edges of the cover.

14. The nipple shield of claim 13, wherein the cover is configured such that a stream of supplement from the supply tube can create a gap and flow under pressure from the open port to the tip section in an unattached area between the cover and the interior surface of the middle section.

15. The nipple shield of claim 13, wherein the interior tip surface is concave and the exterior tip surface is convex.

16. The nipple shield of claim 13, wherein the tip section, the middle section and the lower section are formed of silicone rubber.

17. The nipple shield of claim 13, wherein the tip section, the middle section, the lower section, the supply tube, and the cover are formed of silicone rubber.

18. The nipple shield of claim 13, wherein the cover is formed of a flap of material that is molded contiguously with at least the lower section, and wherein the molded flap is subsequently attached to the middle section along two side edges of the cover.
